(19) United States
(12) Patent Application Publication
(10) Pub. No.: US 2004/0215257 A1
(43) Pub. Date: Oct. 28, 2004

(54) Ventricular rate stabilization with cardiac resynchronization

(76) Inventors: Geeske Van Oort, Velp (NL); J. W. Van Hove, Schiedam (NL)

Correspondence Address: MEDTRONIC, INC., 710 MEDTRONIC PARKWAY NE, MS-LC340, MINNEAPOLIS, MN, 55432-5604, US

(21) Appl. No.: 10/424,583

(22) Filed: Apr. 25, 2003

(51) Int. Cl. A61N 1/362

(52) U.S. Cl. 607/9

(57) ABSTRACT

An implantable medical device (IMD) selectively switches to a more hemodynamically beneficial pacing mode upon detection of ventricular dysynchrony and/or reduced hemodynamic function during delivery of pacing pulses according to a ventricular rate stabilization algorithm. For example, in some embodiments of the invention, an IMD switches from right ventricular pacing according to a ventricular rate stabilization algorithm, to biventricular pacing according to the algorithm. The biventricular pacing can be provided according to a cardiac resynchronization therapy mode, and can involve use of an intraventricular delay between delivery of pacing pulses to the respective ventricles to improve hemodynamic functioning of a heart. The IMD monitors an electrogram signal to detect ventricular dysynchrony and/or decreased hemodynamic performance of the ventricles. The IMD can detect ventricular dysynchrony based on elongated QRS complex widths. The IMD can detect decreased hemodynamic performance based on shortened Q-T intervals and/or decreased ventricular evoked response amplitudes.

### FIELD OF THE INVENTION

The invention relates to medical devices and, more particularly, to implantable medical devices used for cardiac pacing.

### BACKGROUND OF THE INVENTION

When functioning properly, a heart maintains its own intrinsic rhythm, and is capable of pumping adequate blood throughout a circulatory system. This intrinsic rhythm is a function of intrinsic signals generated by the sinoatrial node, or SA node, located in the upper right atrium. The SA node periodically depolarizes, which in turn causes the atrial heart tissue to depolarize such that right and left atria contract as the depolarization travels through the atrial heart tissue. The atrial depolarization signal is also received by the atrioventricular node, or AV node, which, in turn, triggers a subsequent ventricular depolarization signal that travels through and depolarizes the ventricular heart tissue causing the right and left ventricles to contract.

Some patients, however, have irregular cardiac rhythms, referred to as cardiac arrhythmias. Cardiac arrhythmias result in diminished blood circulation because of diminished cardiac output. Atrial fibrillation is a common cardiac arrhythmia that reduces the pumping efficiency of the heart. Atrial fibrillation is characterized by rapid, irregular, uncoordinated depolarizations of the atria. These depolarizations may not originate from the SA node, but may instead originate from an arrhythmogenic substrate, such as an ectopic focus, within the atrial heart tissue. The reduced pumping efficiency due to atrial fibrillation requires the ventricle to work harder, which is particularly undesirable in sick patients that cannot tolerate additional stresses. As a result of atrial fibrillation, patients must typically limit activity and exercise.

An even more serious problem, however, is the risk that atrial fibrillation may induce irregular ventricular heart rhythms. Irregular atrial depolarization signals associated with atrial fibrillation are received by the AV node and may be conducted to ventricles. During atrial fibrillation, the intervals between ventricular depolarizations vary substantially. Such induced ventricular arrhythmias compromise pumping efficiency even more drastically than atrial arrhythmias and, in some instances, may be life threatening. This phenomenon is referred to as conducted atrial fibrillation, or “conducted AF.”

One mode of treating conducted AF is the delivery of cardiac pacing therapy according to a ventricular rate stabilization (VRS) algorithm. In general, VRS algorithms cause an implantable medical device, e.g., a cardiac pacemaker, to deliver pacing pulses at a rate that tracks and is near to the average intrinsic ventricular rate by adjusting a ventricular escape interval as a function of the average intrinsic ventricular rate. By delivering pacing pulses at a rate that tracks and is near to the average intrinsic ventricular rate, implantable medical devices employing a VRS algorithm reduce the instability of the ventricular rate.

Typically, implantable medical devices that deliver cardiac pacing therapy according to a VRS algorithm deliver pacing pulses via an electrode located in the right ventricle near the apex of the heart. In some cases, delivery of pacing pulses at such a location causes dysynchronous contractions of the ventricles due to the way in which the depolarizations resulting from pacing pulses spread throughout the myocardium without the benefit of the specialized conduction pathways of the heart. This ventricular “dysynchrony” can cause reduced cardiac output, which can, in turn, lead to symptoms of congestive heart failure (CHF). Ventricular dysynchrony can also lead to mitral regurgitation. In such cases, the benefit of stabilizing the ventricular rate can be overshadowed by the negative effects of such pacemaker induced ventricular dysynchrony.

### BRIEF SUMMARY OF THE INVENTION

In general, the invention is directed to techniques for ventricular rate stabilization that address the potential for ventricular dysynchrony to occur and impair cardiac output during the delivery of pacing pulses according to a ventricular rate stabilization algorithm. In particular, an implantable medical device according to the invention selectively switches to a more hemodynamically beneficial pacing mode upon detection of ventricular dysynchrony and/or reduced hemodynamic function during delivery of pacing pulses according to a ventricular rate stabilization algorithm. For example, in some embodiments of the invention, an implantable medical device switches from right ventricular pacing according to a ventricular rate stabilization algorithm, to biventricular pacing according to the algorithm. The biventricular pacing can be provided according to a cardiac resynchronization therapy mode, and can involve use of an intraventricular delay between delivery of pacing pulses to the respective ventricles to improve hemodynamic functioning of a heart.

During delivery of pacing pulses according to a ventricular rate stabilization algorithm, an implantable medical device monitors an electrogram signal to detect ventricular dysynchrony and/or decreased hemodynamic performance of the ventricles. The implantable medical device can detect ventricular dysynchrony based on elongated QRS complex widths. The implantable medical device can detect decreased hemodynamic performance based on shortened Q-T intervals and/or decreased ventricular evoked response amplitudes. According to some embodiments of the invention, an implantable medical device digitally processes the electrogram signal to measure these features of the electrogram signal.

In one embodiment, the invention is directed to a device that includes a first electrode to deliver pacing pulses to a first ventricle of a heart of the patient, and a second electrode to deliver pacing pulses to a second ventricle of the heart. The device also includes a processor. The processor controls delivery of pacing pulses via the first electrode according to a ventricular rate stabilization algorithm, and monitors an electrogram of the patient detected during delivery of pacing pulses via the first electrode according to the ventricular rate stabilization algorithm. The processor further controls delivery of pacing pulses via the second electrode according to the ventricular rate stabilization algorithm based on the electrogram. The processor may control biventricular delivery of pacing pulses via the first and second electrodes based on the electrogram signal, and the biventricular delivery of pacing pulses may be according to a cardiac resynchronization pacing mode and with an interventricular delay between delivery of pacing pulses via the first and second electrodes.

In another embodiment, the invention is directed to a method in which pacing pulses are delivered to a first ventricle of a heart of a patient according to a ventricular rate stabilization algorithm. An electrogram signal of the patient is monitored during delivery of pacing pulses to the first ventricle according to the ventricular rate stabilization algorithm, and pacing pulses are delivered to a second ventricle of the heart according to the ventricular rate stabilization algorithm based on the electrogram signal.

In another embodiment, the invention provides a computer-readable medium that comprises program instructions. The program instructions cause a programmable processor to control delivery of pacing pulses to a first ventricle of a heart of a patient via a first electrode according to a ventricular rate stabilization algorithm, and monitor an electrogram signal of the patient detected during delivery of pacing pulses via the first electrode according to the ventricular rate stabilization algorithm. The program instructions further cause a programmable processor to control delivery of pacing pulses to a second ventricle via a second electrode according to the ventricular rate stabilization algorithm based on the electrogram signal.

### BRIEF DESCRIPTION OF THE DRAWINGS

FIG. 1 is a conceptual diagram illustrating an exemplary implantable medical device implanted in a patient that selectively switches to biventricular pacing during delivery of pacing pulses according to a ventricular rate stabilization algorithm.

FIG. 2 is conceptual diagram further illustrating the implantable medical device of FIG. 1 and the heart of the patient.

FIG. 3 is a functional block diagram of the implantable medical device of FIG. 1.

FIG. 4 is a timing diagram illustrating example electrogram signals that may be processed by the implantable medical device of FIG. 1 to detect ventricular dysynchrony and/or reduced hemodynamic function during delivery of pacing pulses according to a ventricular rate stabilization algorithm.

FIG. 5 is a flow chart illustrating an example operation of the implantable medical device of FIG. 1 that switches to biventricular pacing upon detection of ventricular dysynchrony and/or reduced hemodynamic function during delivery of pacing pulses according to a ventricular rate stabilization algorithm.

### DETAILED DESCRIPTION OF THE INVENTION

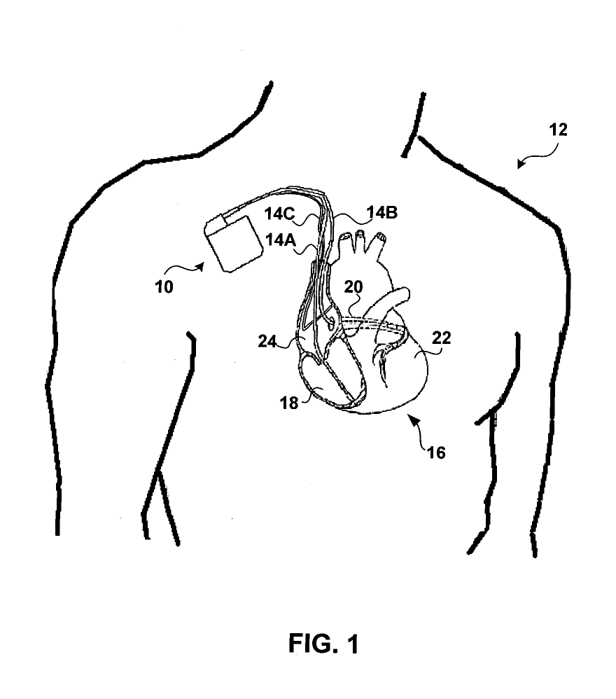

FIG. 1 is a conceptual diagram illustrating an exemplary implantable medical device (IMD) 10 implanted in a patient 12. According to the invention, IMD 10 selectively switches to biventricular delivery of pacing pulses during periods of pacing according to a ventricular rate stabilization (VRS) algorithm in order to improve the hemodynamic performance of the heart 16 of patient 12. IMD 10, as shown in FIG. 1, takes the form of a multi-chamber cardiac pacemaker.

In the exemplary embodiment illustrated in FIG. 1, IMD 10 includes leads 14A, 14B and 14C (collectively “leads 14”) that extend into heart 16. More particularly, right ventricular (RV) lead 14A extends through one or more veins (not shown), the superior vena cava (not shown), and right atrium 24, and into right ventricle 18. Left ventricular (LV) coronary sinus lead 14B extends through the veins, the vena cava, right atrium 24, and into the coronary sinus 20 to a point adjacent to the free wall of left ventricle 22 of heart 16. Right atrial (RA) lead 14C extends through the veins and vena cava, and into the right atrium 24 of heart 16.

IMD 10 senses electrical signals attendant to the depolarization and repolarization of heart 16, and provides pacing pulses via electrodes (not shown) located on leads 14. IMD 10 can also provide cardioversion or defibrillation pulses via electrodes located on leads 14. The sense/pace electrodes located on leads 14 may be unipolar or bipolar, as is well known in the art.

During periods of conducted atrial fibrillation, IMD 10 delivers pacing pulses according a VRS algorithm to stabilize the ventricular rate. IMD 10 may initially deliver VRS pacing to right ventricle 24 via RV lead 14A. As will be described in greater detail below, IMD 10 receives a signal, i.e., an electrogram, that represents electrical activity within heart 16 during pacing according to the VRS algorithm, and processes the signal to detect dysynchrony of the contraction of ventricles 18 and 23 and/or decrease hemodynamic function of the ventricles.

For example, IMD 10 may measure the widths of QRS complexes within the electrogram signal to detect ventricular dysynchrony, or may measure the lengths of Q-T intervals or the amplitudes of R-waves resulting from delivery of pacing pulses, i.e., evoked ventricular responses, to detect decreased ventricular hemodynamic function. QRS complex widths over 150 ms are generally a result of ventricular dysynchrony. Ventricular evoked response amplitudes less than 0.4 mV are indicative of reduced hemodynamic function, e.g., reduced stroke volume, and shortened Q-T intervals indicate increased sympathetic drive resulting from inadequate cardiac output.

Upon detection of ventricular dysynchrony and/or decreased ventricular hemodynamic function, IMD 10 switches to biventricular delivery of pacing pulses according to the VRS algorithm, i.e., delivery of pacing pulses to ventricles 18 and 22 via leads 14A and 14B. Biventricular delivery of pacing pulses may reduce ventricular dysynchrony, and consequently improve the hemodynamic performance of ventricles 18 and 22.

The configuration of MD 10 and leads 14 illustrated in FIG. 1 is merely exemplary. IMD 10 may be coupled any number of leads 14 that extend to a variety of positions within or outside of heart 16. For example, at least some of leads 14 may be epicardial leads. Further, IMD 10 need not be implanted within patient 12, but may instead be coupled with subcutaneous leads 14 that extend through the skin of patient 12 to a variety of positions within or outside of heart 16.

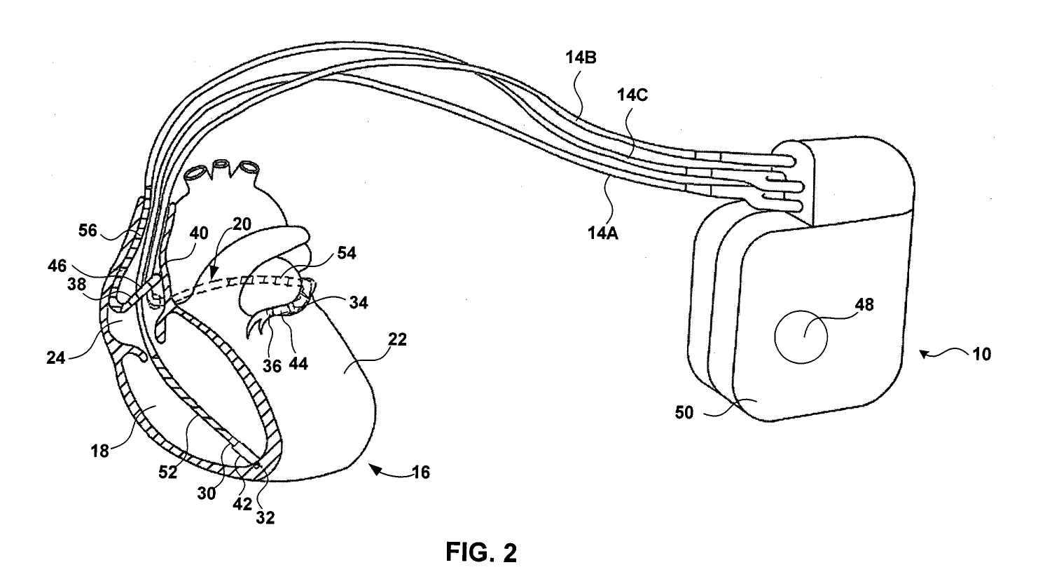

FIG. 2 is conceptual diagram further illustrating IMD 10 and heart 16 of patient 12. Each of leads 14 includes an elongated insulative lead body carrying a number of concentric coiled conductors separated from one another by tubular insulative sheaths. Located adjacent distal end of leads 14A, 14B and 14C are bipolar electrodes 30 and 32, 34 and 36, and 38 and 40 respectively. Electrodes 30, 34 and 38 may take the form of ring electrodes, and electrodes 32, 36 and 40 may take the form of extendable helix tip electrodes mounted retractably within insulative electrode heads 42, 44 and 46, respectively. Each of the electrodes 30-40 is coupled to one of the coiled conductors within the lead body of its associated lead 14.

Sense/pace electrodes 30, 32, 34, 36, 38 and 40 sense electrical signals attendant to the depolarization and repolarization of heart 16. The electrical signals are conducted to IMD 10 via leads 14. Sense/pace electrodes 30, 32, 34, 36, 38 and 40 further deliver pacing pulses to cause depolarization of cardiac tissue in the vicinity thereof. IMD 10 may also include one or more indifferent housing electrodes, such as housing electrode 48, formed integral with an outer surface of the hermetically sealed housing 50 of IMD 10. Any of electrodes 30, 32, 34, 36, 38 and 40 may be used for unipolar sensing or pacing in combination with housing electrode 48.

The invention is not limited to the sense/pace electrode locations illustrated in FIG. 2. For example, in the example embodiment illustrated in FIG. 2, tip electrode 32 of RV lead 14A is disposed in the apical region of right ventricle 18. However, in other embodiments, tip electrode 32 may be located near the pulmonary artery outflow tract (not shown) or the bundle of His. Such alternative locations may provide a more synchronized, and thus hemodynamically beneficial, contraction of ventricles 18 and 22 through delivery of pacing at a single location by delivering pulses near the specialized conduction system of heart 16.

Leads 14A, 14B and 14C may also, as shown in FIG. 2, include elongated coil electrodes 52, 54 and 56, respectively. IMD 10 may deliver defibrillation or cardioversion shocks to heart 16 via defibrillation electrodes 52-56. Defibrillation electrodes 52-56 may be fabricated from platinum, platinum alloy or other materials known to be usable in implantable defibrillation electrodes, and may be about 5 cm in length.

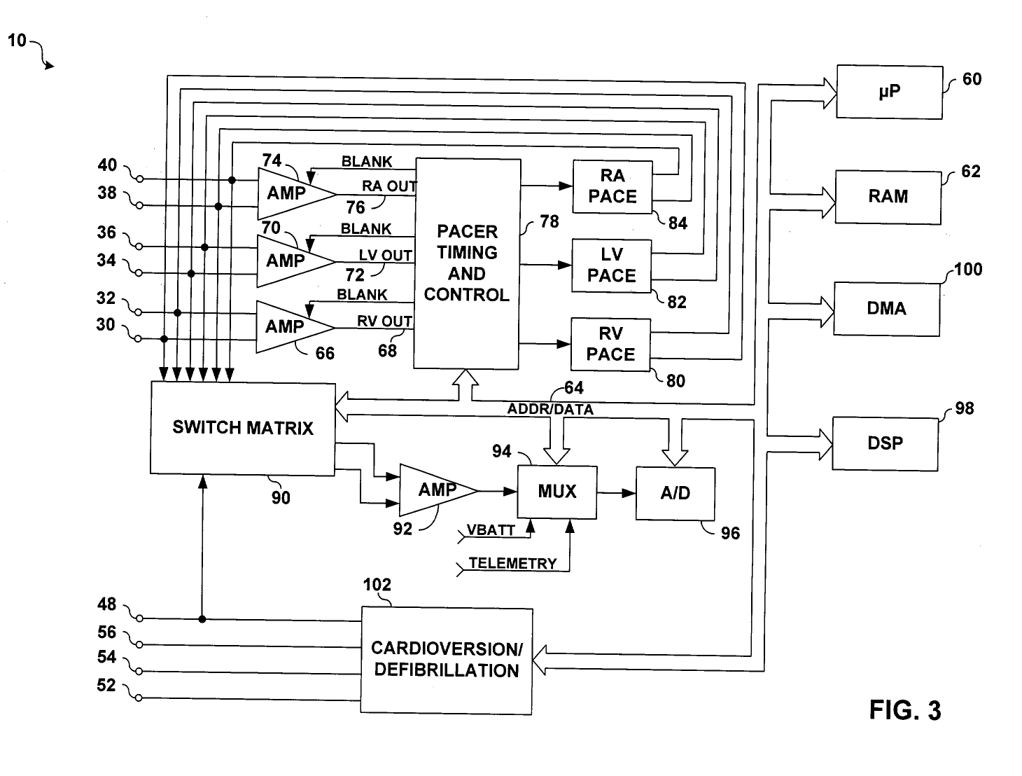

FIG. 3 is a functional block diagram of IMD 10. As shown in FIG. 3, IMD 10 may take the form of a multi-chamber pacemaker-cardioverter-defibrillator (PCD) having a microprocessor-based architecture. However, this diagram should be taken as exemplary of the type of device in which various embodiments of the present invention may be embodied, and not as limiting, as it is believed that the invention may be practiced in a wide variety of device implementations, including devices that provide pacing therapies but do not provide cardioverter and/or defibrillator functionality.

IMD 10 includes a microprocessor 60. Microprocessor 60 may execute program instructions stored in a memory, e.g., a computer-readable medium, such as a ROM (not shown), EEPROM (not shown), and/or RAM 62. Program instruction stored in a computer-readable medium and executed by microprocessor 60 control microprocessor 60 to perform the functions ascribed to microprocessor 60 herein. Microprocessor 60 may be coupled to, e.g., communicate with and/or control, various other components of IMD 10 via an address/data bus 64.

IMD 10 detects atrial and ventricular depolarizations. Electrodes 30 and 32 are coupled to amplifier 66, which may take the form of an automatic gain controlled amplifier providing an adjustable sensing threshold as a function of the measured R-wave amplitude. A signal is generated on RV out line 68 whenever the signal sensed between electrodes 30 and 32 exceeds the present sensing threshold. Electrodes 34 and 36 are coupled to amplifier 70, which also may take the form of an automatic gain controlled amplifier providing an adjustable sensing threshold as a function of measured R-wave amplitude. A signal is generated on LV out line 72 whenever the signal sensed between electrodes 34 and 36 exceeds the present sensing threshold. Electrodes 38 and 40 are coupled to amplifier 74, which may take the form of an automatic gain controlled amplifier providing an adjustable sensing threshold as a function of the measured P-wave amplitude. A signal is generated on RA out line 76 whenever the signal between electrodes 38 and 40 exceeds the present sensing threshold.

IMD 10 paces heart 16. Pacer timing/control circuitry 78 preferably includes programmable digital counters which control the basic time intervals associated with modes of pacing. Circuitry 78 also preferably controls escape intervals associated with pacing. In the exemplary biventricular pacing environment, pacer timing/control circuitry 78 controls the ventricular escape interval that is used to time pacing pulses delivered to one or both of ventricles 18 and 22, and, where cardiac resynchronization therapy (CRT) pacing is provided, may control an interval between delivery of pulses to ventricles 18 and 22.

Intervals defined by pacing circuitry 78 may also include the refractory periods during which sensed R-waves and P-waves are ineffective to restart timing of the escape intervals, and the pulse widths of the pacing pulses. The durations of these intervals are determined by microprocessor 60 in response to data stored in RAM 62, and are communicated to circuitry 78 via address/data bus 64. Pacer timing/control circuitry 78 also determines the amplitude of the cardiac pacing pulses under control of microprocessor 60.

Microprocessor 60 operates as an interrupt driven device, and is responsive to interrupts from pacer timing/control circuitry 78 corresponding to the occurrence of sensed P-waves and R-waves, i.e., paced atrial and ventricular depolarizations, and corresponding to the generation of cardiac pacing pulses. Those interrupts are provided via data/address bus 66. Any necessary mathematical calculations to be performed by microprocessor 60 and any updating of the values or intervals controlled by pacer timing/control circuitry 78 take place following such interrupts.

In accordance with the selected mode of pacing, pacer timing/control circuitry 78 triggers generation of pacing pulses by one or more of pacer output circuits 80, 82 and 84, which are coupled to electrodes 30 and 32, 34 and 36, and 38 and 40, respectively. Output circuits 80, 82 and 84 may be pulse generation circuits known in the art, which include capacitors and switches for the storage and delivery of energy as a pulse. Pacer timing/control circuitry 78 resets escape interval counters upon detection of R-waves or P-waves as indicated by signals on lines 68, 72 and 76, or generation of pacing pulses, and thereby control the basic timing of cardiac pacing functions.

For example, when microprocessor 60 indicates pacing according to a VVI mode, pacer timing/control circuitry controls output circuit 80 to deliver pacing pulses via RV lead 14A (FIGS. 1 and 2) and electrodes 30 and 32 upon expiration of a ventricular escape interval provided by microprocessor 60. The ventricular escape interval can be an A-V interval timed from a sensed P-wave indicated on line 76 or a pace delivered to right atrium 24, or can be timed from the previous ventricular depolarization, e.g., a sensed R-wave indicated on either of lines 68 and 72 or a pace delivered to right ventricle 18. In either case, circuitry 78 resets the ventricular escape interval upon detection of an intrinsic R-wave on either of lines 68 and 72, or upon its expiration and the resulting delivery of a pacing pulse.

IMD 10 can detect conducted atrial fibrillation, and delivers pacing pulses according to a VRS algorithm based on detection of conducted atrial fibrillation. IMD 10 may employ any of a number of techniques known in the art for detecting conducted atrial fibrillation. For example, microprocessor 60 may detect conducted atrial fibrillation based on the variability of the ventricular rate as determined based on R-wave indications received from pacer timing/control circuitry 78. Where patient 12 is believed to have intact A-V conduction, microprocessor 60 may simply detect atrial fibrillation based on a rapid increase in the rate of P-wave indication received from circuitry 78, and direct circuitry 78 to provide VRS pacing based on the detected atrial fibrillation. The invention is not limited to any particular technique for detecting conducted atrial fibrillation.

When conducted atrial fibrillation is detected, microprocessor 60 controls delivery of pacing pulses to right ventricle 18 according to a VRS algorithm. Microprocessor 60 may employ any of a number of known VRS algorithms, and the invention is not limited to any particular algorithm or type of algorithm. An exemplary VRS algorithm that may be employed by microprocessor 60 is disclosed in an article by Duckers, H. J., et al., entitled “Effective use of a novel rate-smoothing algorithm in atrial fibrillation by ventricular pacing,” European Heart Journal, December 1997, pp. 1951-1955. In general, according to such a VRS algorithm, microprocessor 60 monitors the average ventricular rate based on R-wave and ventricular pace indications received from pacer timing/control circuitry 78, and adjusts the ventricular escape interval to provide a pacing rate at or near the average ventricular rate. Pacing at or near the average ventricular rate is believed to regularize the ventricular rate.

During delivery of pacing pulses according to a VRS algorithm, microprocessor 60 monitors an electrogram signal to detect ventricular dysynchrony and/or decreased ventricular hemodynamic function. IMD 10 may, as shown in FIG. 3, include circuitry to digitally analyze the electrogram signal to facilitate monitoring of the electrogram signal by microprocessor 60. Switch matrix 90 is used to select which of the available electrodes 30-40 and 48 are coupled to wide band (0.5-200 Hz) amplifier 92 for use in digital signal analysis. As will be described in greater detail below, any of a number of potential combinations of these electrodes may be used, so long as the signal provided by the combination allows for identification and measurement of features of the electrogram signal that indicate ventricular dysynchrony and/or decreased ventricular hemodynamic function. Selection of electrodes is controlled by microprocessor 60 via data/address bus 64, and the selections may be varied as desired.

The analog signal derived from the selected electrodes and amplified by amplifier 92 is provided to multiplexer 94, and thereafter converted to a multi-bit digital signal by A/D converter 96. A digital signal processor (DSP) 98 may process the multi-bit digital signal to measure QRS widths, Q-T intervals, and/or ventricular evoked response amplitudes, as will be described in greater detail below. In some embodiments, the digital signal may be stored in RAM 62 under control of direct memory access circuit 100 for later analysis by DSP 98. Although IMD 10 is described herein as having separate processors, microprocessor 60 may perform both the functions ascribed to it herein and digital signal analysis functions ascribed to DSP 98 herein. Moreover, although described herein in the context of microprocessor based PCD embodiment IMD 10, the invention may be embodied in various implantable medical devices that include one or more processors, which may be microprocessors, DSPs, field programmable gate arrays (FPGAs), application specific integrated circuits (ASICs), or other digital logic circuits.

The QRS widths, Q-T intervals, or evoked response amplitudes measured by DSP 98 may be stored in RAM 62 where they may be retrieved for analysis by microprocessor 60. Based on the analysis of one or more of these features, which will be described in greater detail below, microprocessor 60 detects ventricular dysynchrony and/or decreased ventricular hemodynamic function. When ventricular dysynchrony and/or decreased ventricular hemodynamic function is detected, microprocessor 60 directs pacer timing/control circuitry 78 to control delivery of pacing pulses according to the VRS algorithm biventricularly, i.e., circuitry 78 controls both of output circuits 80 and 82 to generate and deliver pacing pulses via electrodes 30-36 when a pace is indicated by expiration of the ventricular escape interval.

Biventricular delivery of pacing pulses according to the VRS algorithm may be according to a CRT pacing mode. For example, microprocessor 60 may provide pacer timing/control circuitry 78 with an atrioventricular (A-V) and interventricular (V-V) delay. Circuitry 78 may control delivery of a pacing pulse to a first one of ventricles 18 and 22 upon expiration of the A-V delay, and a second one of ventricles 18 and 22 upon expiration of the V-V delay after delivery of the pacing pulse to the first one of ventricle 18 and 22. Microprocessor 60 may adjust the values of the A-V and V-V delays based on feedback received from sensors that indicates the hemodynamic functioning of ventricles 18 and 22, as is known in the art.

IMD 10 may detect ventricular and/or atrial tachycardias or fibrillations of heart 16 using tachycardia and fibrillation detection techniques and algorithms known in the art. For example, the presence of a ventricular or atrial tachycardia or fibrillation may be confirmed by detecting a sustained series of short R-R or P-P intervals of an average rate indicative of tachycardia, or an unbroken series of short R-R or P-P intervals. IMD 10 is also capable of delivering one or more anti-tachycardia pacing (ATP) therapies to heart 16, and cardioversion and/or defibrillation pulses to heart 16 via one or more of electrodes 48, 52, 54 and 56.

Electrodes 48, 52, 54 and 56, are coupled to a cardioversion/defibrillation circuit 90, which delivers cardioversion and defibrillation pulses under the control of microprocessor 60. Circuit 90 may include energy storage circuits such as capacitors, switches for coupling the storage circuits to electrodes 48, 52, 54 and 5, and logic for controlling the coupling of the storage circuits to the electrodes to create pulses with desired polarities and shapes. Microprocessor 60 may employ an escape interval counter to control timing of such cardioversion and defibrillation pulses, as well as associated refractory periods.

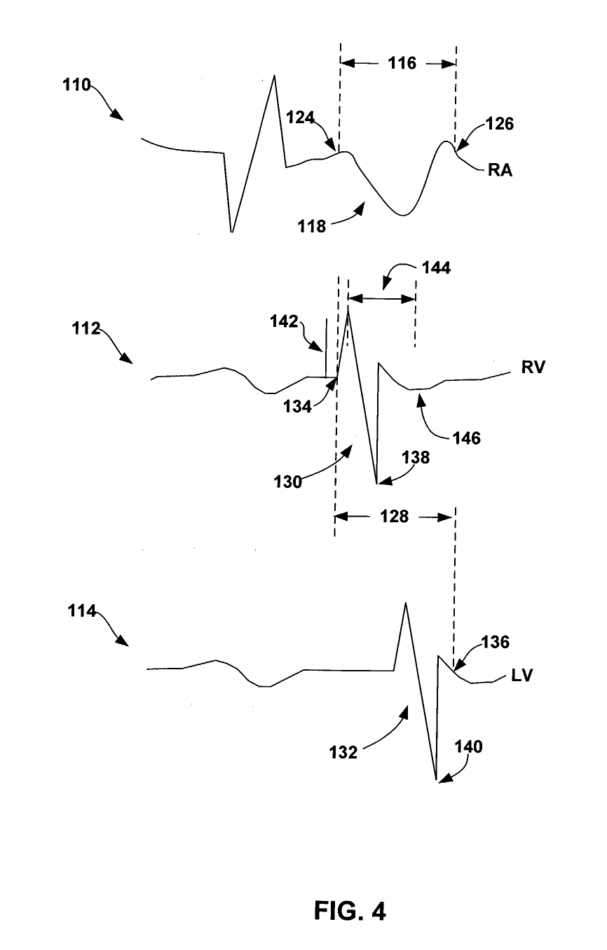

FIG. 4 is a timing diagram illustrating example electrogram (EGM) signals that may be processed by IMD 10 to detect ventricular dysynchrony and/or reduced hemodynamic function during delivery of pacing pulses according to a VRS algorithm. Signal 110 is a right atrial EGM. IMD 10 may digitally process right atrial EGM 110 to measure a width 116 of far-field QRS complex 118 in order to detect ventricular dysynchrony. Right atrial EGM 110 may be detected using electrodes 38 and 40 of RA lead 14C in a bipolar configuration, or one of electrodes 38 and 40 and housing electrode 48 in a unipolar configuration.

Where IMD 10 measures QRS widths to detect ventricular dysynchrony, it is generally preferred that IMD 10 process signals that include far-field QRS complexes, such as right atrial EGM 110. Processing these signals is preferred because such signals include QRS complexes that are more “global” in that they reflect depolarization of both ventricles 18, 22, and thus indicate dysynchrony between depolarization of the ventricles. In addition to atrial EGM signal 110, IMD 10 may detect signals that include far-field QRS complexes using two or more housing electrodes 48.

In order to measure QRS complex width 116, DSP 98 first identifies far-field QRS complex 118 within signal 110. DSP 98 may identify QRS complex 118 within signal 110 by any methods known in the art. For example, DSP 98 may receive indications of the occurrence of an R-wave 120 or 122 from pacer timing/control circuit 78, and identify QRS complex 118 based on these indications. As another example, DSP 98 may identify QRS complex 118 by detecting a number of threshold-crossings of the digital signal provided by A/D converter 96, or zero-crossings of the first derivative of the digital signal occurring within a time window. As yet another example, DSP 98 may detect far-field QRS complexes 118 within signal 110 using techniques described in commonly assigned U.S. Pat. No. 6,029,087, to Wohlgemuth, and titled “Cardiac Pacing System With Improved Physiological Event Classification Based on DSP” (“Wohlgemuth '087 Patent”).

DSP 98 may measure width 116 as a period of time from a beginning point 124 to an ending point 126. DSP 98 may identify beginning point 124 and ending point 126 as threshold-crossings of the digital signal or zero-crossings of the first derivative of the digital signal.

DSP 98 may also measure a “global” QRS width 128 via right ventricular EGM signal 112 and left ventricular EGM 114. Right and left ventricular EGMs 112 and 114 are detected via RV lead 14C and LV coronary sinus lead 14B, respectively, using bipolar electrode pairs 30, 32 and 34, 36, or one electrode from each pair and housing electrode 48 in a unipolar configuration. QRS complexes 130 and 132 may be detected by any of the methods described above with reference to far-field QRS complex 118, and width 128 is measured from the first beginning point to the last ending point of QRS complexes 118 and 120, e.g., beginning point 134 of QRS complex 130 to ending point 136 of QRS complex 132 in the illustrated example. Beginning and ending points 134 and 136 may be identified by any of the method described above with reference to beginning and ending points 124 and 126. As can be seen in FIG. 4, QRS width 118 represents the width of the overall depolarization of ventricles 18 and 22.

Microprocessor 60 may detect decreased ventricular hemodynamic function based on decreased evoked response amplitudes. DSP 98 may also measure ventricular evoked response amplitudes, i.e., the amplitudes of one or both of R-waves 138 and 140 or signals 112 and 114, respectively, resulting from delivery of a pacing pulse 142. In general, it is preferred that DSP 98 measure the amplitude of the R-wave 138, 140 from the lead 14A, 14B that delivered pacing pulse 142.

Microprocessor 60 may also detect decreased ventricular hemodynamic function based on shortened Q-T intervals. DSP 98 may, for example, measure a Q-T interval 144 within EGM signal 112. In some embodiments, DSP 98 receives an indication of delivery of a pacing pulse 142 from pacer timing/control circuitry 78, and measures Q-T interval 144 as the period of time from delivery of pacing pulse 142 to detection of T-wave 146 within the digital signal provided by A/D converter 96. T-wave 146 may, for example, be detected using techniques described in the above-referenced Wohlgemuth '087 Patent.

For ease of illustration, only a portion of each of EGM signals 110-114 representing a single cardiac cycle of heart 16 is shown in FIG. 4. However, it is understood that DSP 98 measures multiple QRS complex widths, Q-T intervals and/or evoked response amplitudes over multiple cardiac cycles.

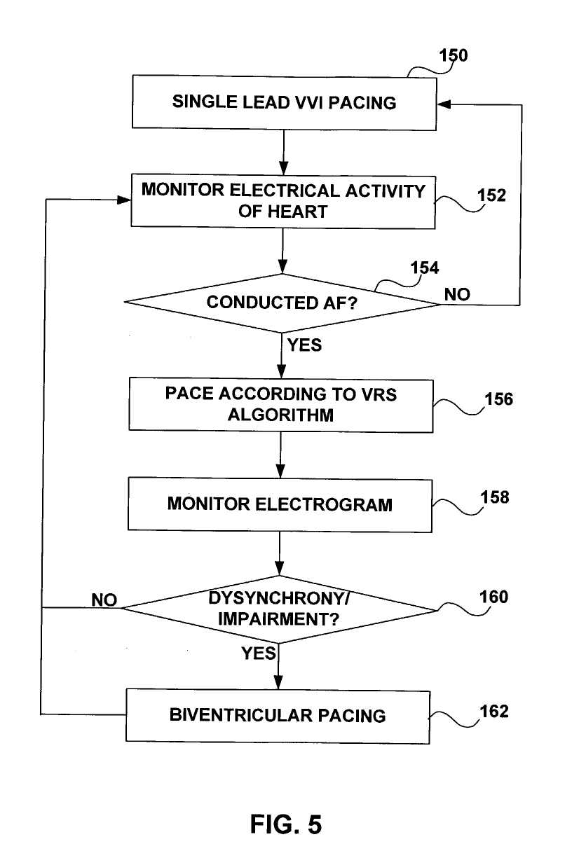

FIG. 5 is a flow chart illustrating an example operation of IMD 10 to switch to biventricular pacing upon detection of ventricular dysynchrony and/or reduced hemodynamic function during delivery of pacing pulses according to a VRS algorithm. Initially, in the absence of conducted atrial fibrillation, IMD 10 delivers pacing pulses to right ventricle 18 according to a VVI mode, which may be a rate responsive, e.g., VVIR, mode (150). IMD 10 monitors electrical activity of heart 16 to detect conducted atrial fibrillation (152). As described above, microprocessor 60 may detect conducted atrial fibrillation by detecting increased variability in the ventricular rate, i.e., increased variability in intervals between R-waves indications received from pacer timing/control circuitry 78, or by simply detecting atrial fibrillation based on an increased rate of P-wave indications received from circuitry 78.

If microprocessor 60 detects conducted atrial fibrillation (154), microprocessor 60 directs circuitry 78 to control delivery of pacing pulses to right ventricle 18 according to a VRS algorithm, e.g., provides circuitry 78 with a ventricular escape interval determined based on the average ventricular rate (156). During delivery of pacing pulses according to the VRS algorithm, microprocessor 60 monitors one or more electrogram signals, as described above, in order to detect ventricular dysynchrony and/or hemodynamic impairment (158).

In particular, microprocessor 60 receives measured values of one or more of QRS complex widths, evoked response amplitudes, or Q-T intervals from DSP 98. Microprocessor 60 may detect ventricular dysynchrony based on the measured values of QRS complex widths exceeding a threshold value. Microprocessor 60 may require a single measure QRS complex width, a number of consecutive measured QRS complex widths, a number of QRS complex widths within a window, or an average QRS complex width over a period of time, to exceed the threshold before determining that ventricular dysynchrony is occurring.

Similarly, microprocessor 60 may detect impaired ventricular hemodynamic function, which can result from ventricular dysynchrony, by comparison of one or more measured evoked response amplitudes and/or Q-T intervals to a threshold value. Microprocessor 60 may also detect impaired ventricular hemodynamic function by comparison of the rate of change of one or more of these measured values over time to a threshold values. Threshold values for the detection of ventricular dysynchrony and/or impaired ventricular hemodynamic function can be stored in RAM 62 (FIG. 3).

Because biventricular delivery of pacing pulses consumes more energy from the power source, e.g., battery, of IMD 10, IMD 10 initially delivers pacing pulses to a single ventricle, e.g., right ventricle 18 via lead 14A, according to the VRS algorithm. However, if ventricular dysynchrony and/or impaired ventricular hemodynamic function are detected during pacing according to the VRS algorithm (160), microprocessor 60 controls biventricular delivery of pacing pulses according to the VRS algorithm in order to improve ventricular synchrony and hemodynamic functioning (162). As discussed above, biventricular delivery of pacing pulses may include pacing according to a cardiac resynchronization therapy mode.

In order to monitor the effectiveness of biventricular pacing, IMD 10 continues to monitor the one or more EGM signals during delivery of biventricular pacing. If the measured values drop below threshold, or are below threshold for a sufficient period of time, IMD 10 may resume right ventricular pacing. Further, in some embodiments, IMD 10 may modify parameters of biventricular pacing, such as A-V or V-V intervals, or resume right ventricular pacing if, after a period of time, it is determined that biventricular pacing with the current parameters has not improved hemodynamic function or ventricular synchrony.

Similarly, IMD 10 may monitor the effectiveness of pacing according to the VRS algorithm, and may select a new algorithm, modify parameters of the VRS algorithm, or cease delivery of pacing pulse according to the VRS algorithm based on the effectiveness. For example, based on R-wave indications received from pacer timing/control circuitry 78, microprocessor 60 may determine whether conducted atrial fibrillation has ended, or whether the VRS algorithm has improved ventricular stability or undesirably increased the average ventricular rate. Further microprocessor 60 may assess the effectiveness of the VRS algorithm based on hemodynamic performance, by, for example, monitoring Q-T interval lengths within a ventricular electrogram.

A number of embodiments of the invention have been described. However, one skilled in the art will appreciate that the invention can be practiced with embodiments other than those disclosed. For example, the invention is not limited to IMD embodiments that switch to biventricular pacing upon detection of ventricular dysynchrony and/or impaired hemodynamic function. Instead, in some embodiments, an IMD may switch from pacing at a first location to a pacing at a second location. For example, an IMD may switch from right ventricular pacing to left ventricular pacing, which may allow for more synchronized and hemodynamically effective ventricular contraction, upon detection of ventricular dysynchrony and/or impaired ventricular hemodynamic function. The disclosed embodiments are presented for purposes of illustration and not limitation, and the invention is limited only by the claims that follow.

What is claimed is:

1. An implantable medical device comprising:
a first electrode to deliver pacing pulses to a first ventricle of a heart of a patient;
a second electrode to deliver pacing pulses to a second ventricle of the heart; and
a processor to control delivery of pacing pulses via the first electrode according to a ventricular rate stabilization algorithm, monitor an electrogram signal of the patient detected during delivery of pacing pulses via the first electrode according to the ventricular rate stabilization algorithm, and control delivery of pacing pulses via the second electrode according to the ventricular rate stabilization algorithm based on the electrogram signal.

2. The implantable medical device of claim 1, wherein the processor controls biventricular delivery of pacing pulses via the first and second electrodes based on the electrogram signal.

3. The implantable medical device of claim 2, wherein the processor controls biventricular delivery of pacing pulses according to a cardiac resynchronization pacing mode and with an interventricular delay between delivery of pacing pulses via the first and second electrodes.

4. The implantable medical device of claim 1, wherein the processor detects ventricular dysynchrony based on the electrogram signal, and controls delivery of pacing pulses via the second electrode based on the detection.

5. The implantable medical device of claim 4, wherein the processor determines the widths of QRS complexes within the electrogram signal, and detects ventricular dysynchrony based on the QRS complex widths.

6. The implantable medical device of claim 1, wherein the processor monitors a feature of the electrogram signal that indicates hemodynamic performance of the heart, and controls delivery of pacing pulses via the second electrode based on the feature.

7. The implantable medical device of claim 6, wherein the processor monitors at least one of evoked R-wave amplitudes and Q-T intervals.

8. The implantable medical device of claim 1, wherein the processor controls delivery of pacing pulses according to a ventricular rate stabilization algorithm by determining an average ventricular rate and adjusting a ventricular escape interval based on the average ventricular rate.

9. The implantable medical device of claim 1, further comprising a housing and a third electrode integral with the housing, wherein the processor monitors an electrogram signal detected via at least one of the first, second and third electrodes.

10. The implantable medical device of claim 1, wherein the first ventricle is a right ventricle of the heart and the second ventricle is a left ventricle of the heart.

11. The implantable medical device of claim 1, wherein the first and second electrodes are intracardiac electrodes.

12. A method comprising:
delivering pacing pulses to a first ventricle of a heart of a patient according to a ventricular rate stabilization algorithm;
monitoring an electrogram signal of the patient during delivery of pacing pulses to the first ventricle according to the ventricular rate stabilization algorithm; and
delivering pacing pulses to a second ventricle of the heart according to the ventricular rate stabilization algorithm based on the electrogram signal.

13. The method of claim 12, wherein delivering pacing pulses to a second ventricle comprises delivering pacing pulses to the first and second ventricles for each indicated ventricular pace.

14. The method of claim 13, wherein delivering pacing pulses to the first and second ventricles comprises delivering pacing pulses according to a cardiac resynchronization pacing mode and with an interventricular delay between delivery of pacing pulses to the first and second ventricles.

15. The method of claim 12, wherein monitoring an electrogram signal of the patient comprises detecting ventricular dysynchrony based on the electrogram signal, and
wherein delivering pacing pulses to the second ventricle based comprises delivering pacing pulses to the second ventricle based on the detection.

16. The method of claim 15, wherein detecting ventricular dysynchrony comprises:
determining the widths of QRS complexes within the electrogram signal; and
detecting ventricular dysynchrony based on the QRS complex widths.

17. The method of claim 12, wherein monitoring an electrogram signal of the patient comprises monitoring a feature of the electrogram signal that indicates hemodynamic performance of the heart, and
wherein delivering pacing pulses to the second ventricle comprises delivering pacing pulses to the second ventricle based on the feature.

18. The method of claim 17, wherein monitoring a feature of the electrogram signal comprises monitoring at least one of evoked R-wave amplitudes and Q-T intervals.

19. The method of claim 12, wherein delivering pacing pulses according to a ventricular rate stabilization algorithm comprises:
determining an average ventricular rate; and
adjusting a ventricular escape interval based on the average ventricular rate.

20. The method of claim 12, wherein the first ventricle is a right ventricle of the heart and the second ventricle is a left ventricle of the heart.

21. A computer-readable medium comprising instructions that cause a programmable processor to:
control delivery of pacing pulses to a first ventricle of a heart of a patient via a first electrode according to a ventricular rate stabilization algorithm;
monitor an electrogram signal of the patient detected during delivery of pacing pulses via the first electrode according to the ventricular rate stabilization algorithm; and
control delivery of pacing pulses to a second ventricle via a second electrode according to the ventricular rate stabilization algorithm based on the electrogram signal.

22. The computer-readable medium of claim 21, wherein the instructions that cause a programmable processor to control delivery of pacing pulses to a second ventricle comprises instructions that cause a programmable processor to control biventricular delivery of pacing pulses to the first and second ventricles via the first and second electrodes based on the electrogram signal.

23. The computer-readable medium of claim 22, wherein the instructions that cause a programmable processor to control biventricular delivery of pacing pulses comprise instructions that cause a programmable processor to control biventricular delivery of pacing pulses according to a cardiac resynchronization pacing mode and with an interventricular delay between delivery of pacing pulses via the first and second electrodes.

24. The computer-readable medium of claim 21, wherein the instructions that cause a programmable processor to monitor an electrogram signal comprise instructions that cause a programmable processor to detect ventricular dysynchrony based on the electrogram signal, and
wherein the instruction that cause a programmable processor to control delivery of pacing pulses to the second ventricle via the second electrode comprise instructions that cause a programmable processor to control delivery of pacing pulses to the second ventricle via the second electrode based on the detection.

25. The computer-readable medium of claim 24, wherein the instructions that cause a programmable processor to detect ventricular dysynchrony comprise instructions that cause a programmable processor to:
determine the widths of QRS complexes within the electrogram signal; and
detect ventricular dysynchrony based on the QRS complex widths.

26. The computer-readable medium of claim 21, wherein the instructions that cause a programmable processor to monitor an electrogram signal comprise instructions that cause a programmable processor to monitor a feature of the electrogram signal that indicates hemodynamic performance of the heart, and
wherein the instruction that cause a programmable processor to control delivery of pacing pulses to the second ventricle via the second electrode comprise instructions that cause a programmable processor to control delivery of pacing pulses to the second ventricle via the second electrode based on the feature.

27. The computer-readable medium of claim 26, wherein the instructions that cause a programmable processor to monitor a feature of the electrogram signal comprise instructions that cause a programmable processor to monitor at least one of evoked R-wave amplitudes and Q-T intervals.

28. The computer-readable medium of claim 21, wherein the instructions that cause a programmable processor to deliver pacing pulses according to a ventricular rate stabilization algorithm comprise instructions that cause a programmable processor to:
determine an average ventricular rate; and
adjust a ventricular escape interval based on the average ventricular rate.

29. The computer-readable medium of claim 21, wherein the first ventricle is a right ventricle of the heart and the second ventricle is a left ventricle of the heart.
